(19) United States
(12) Patent Application Publication
(10) Pub. No.: US 2025/0312306 A1
(43) Pub. Date: Oct. 9, 2025

(54) Concentrate comprising a semiochemical composition

(71) Applicant: SIGNS, APT (FR)

(72) Inventors: Maréva REBOUL, APT (FR); Mélinda TEP, APT (FR); Patrick PAGEAT, APT (FR)

(21) Appl. No.: 19/036,231

(22) Filed: Jan. 24, 2025

(30) Foreign Application Priority Data
Apr. 5, 2024 (FR) 2403567

(51) Int. Cl. A61K 31/34; A61K 31/22; A61K 31/225

(52) U.S. Cl. CPC A61K 31/34; A61K 31/22; A61K 31/225

(57) ABSTRACT

The invention relates to the field of semiochemical products, and describes in particular a composition in the form of a concentrate of these products that can be diluted in water, comprising a semiochemical composition, in particular a lipophilic and water-dispersible semiochemical composition, and its preparation process. The invention also relates to the non-therapeutic use of the diluted composition according to the invention for topical, in particular cutaneous, administration to an animal or human.

### TECHNICAL FIELD OF THE INVENTION

The invention relates to the field of semiochemical products, and describes in particular a composition in the form of a concentrate of these products that can be diluted in water, comprising a semiochemical composition, in particular a lipophilic and water-dispersible semiochemical composition, as well as its preparation process. The invention also relates to the non-therapeutic use of the diluted composition according to the invention for topical, in particular cutaneous, administration to an animal or human.

### PRIOR STATE OF THE ART

Semiochemicals according to the invention correspond to mixtures of several chemical compounds, described for the first time in particular by Silverstein, R.M. (1977). Semiochemicals are chemical substances released by a plant or animal that provoke a non-therapeutic behavioural or physiological response in another organism. When the semiochemical affects an individual of the same species (“intra-specific semiochemical product”), it is called a pheromone (Wyatt T.D., 2003). There are several types of pheromones that enable different types of behavioural interaction or communication, such as soothing pheromones, sex pheromones, territorial marking pheromones and warning pheromones. When the semiochemical product affects an individual of a different species (“interspecific semiochemical product”), it is called an allelochemical substance. Among allelochemical substances, an allomone is an allelochemical substance that triggers a reaction in the target species, this reaction being favourable to the species that releases the allomone (the opposite is a kairomone).

Since 1995, specialities whose active ingredients are semiochemical agents have been marketed. Some of their uses require the product to be deposited on the animal's skin, for example to obtain the desired effects on an animal in an open environment or to protect it from pests such as parasites or biting insects, or on human skin. The physico-chemical constraints of semiochemical components, and in particular their volatility and insolubility in water, have led to the use of excipients such as ethoxydiglycol in marketed specialities. The use of this type of excipient is now increasingly restricted by current regulations, but also by the general public, tending to reject them because of the way they are synthesised and their potential direct and environmental toxicity. It therefore appeared necessary to develop aqueous formulations of semiochemical compounds in different galenic forms.

### DISCLOSURE OF THE INVENTION

The aim of the invention is to develop a composition based on semiochemical products (also referred to as compounds, active ingredients or agents) in a concentrated form enabling the preparation of a new galenic form, preferably non-toxic and environmentally friendly, in particular a composition in the form of a concentrate diluted in water, comprising a semiochemical composition. A composition in the form of a concentrate enables an end user to obtain a ready-to-use composition following a dilution step, in particular a dilution of the concentrate in water. This dilution is easy to achieve and can be carried out on the spot, before or at the time of use, preferably extemporaneously, according to the final quantities and concentrations desired by the end user, thus reducing the weight of the composition in the form of a concentrate distributed and the volume of water, particularly large quantities of water, for transport, and enabling it to be used on large surfaces, for example livestock buildings. The composition in the form of a concentrate has a long shelf life (at least 2 years). A non-therapeutic use of said composition in the form of a concentrate, after dilution, relates to topical, in particular cutaneous, administration of the composition to an animal, in particular poultry such as hens, in particular to the skin of a poultry such as a hen, in particular to prevent infestation by parasites such as the poultry red mite (Dermanyssus sp. or Ornithonyssus sylviarum). Another non-therapeutic use of said composition in the form of a concentrate, after dilution, concerns topical, in particular cutaneous, administration of the composition to an animal, for example birds or mammals, in particular on the skin of birds or mammals, or to a human, in particular on human skin, in particular to prevent infestation or bites by haematophagous invertebrates, in particular haematophagous insects, such as mosquitoes, mites or ticks.

### SUMMARY OF THE INVENTION

The invention is defined by the independent claims. The dependent claims define particular or preferred embodiments of the invention.

According to a first aspect, the invention relates to a composition in the form of a concentrate that can be diluted in water, comprising:
a) at least one polyoxyethylene sorbitan fatty acid ester;
b) a lipophilic and water-dispersible semiochemical composition, in particular a semiochemical composition comprising bis(2-ethylhexyl)adipate and 1-isopropyl-2,2-dimethyltrimethylene diisobutyrate in a content of at least 40% by weight of the composition in the form of a concentrate,
the total sum of the compounds present in the composition in the form of a concentrate being equal to 100%.

According to a second aspect, the invention relates to a process for preparing a composition in the form of a concentrate according to the invention, comprising the following steps:
a) the semiochemical composition, said at least one polyoxyethylene sorbitan fatty acid ester and optionally said at least one additive are mixed, with stirring and at a temperature of between 65° C. and 75° C., preferably at a temperature of 70° C., so as to obtain a homogeneous
b) optionally, a perfume is added;
c) the composition obtained in step a) or b) is recovered in the form of a concentrate.

According to a third aspect, the invention relates to the non-therapeutic use of a composition diluted in water according to the invention for topical administration, in particular cutaneous administration to poultry such as hens, in particular on the skin of a poultry such as a hen, in particular to prevent infestation by parasites such as the poultry red mite (Dermanyssus sp. or Ornithonyssus sylviarum); or for topical, in particular cutaneous, administration to an animal, for example birds or mammals, in particular on the skin of birds or mammals, or to a human, in particular on human skin, in particular to prevent infestation or bites by haematophagous invertebrates, in particular haematophagous insects, such as mosquitoes, mites or ticks.

### DETAILED DESCRIPTION OF THE INVENTION

The invention is now described in more detail and in a non-limiting way in the following description. Unless otherwise stated (e.g., specific mention of v %/v%), all percentages relating to quantities are percentages by mass. Any reference to ranges of values in the description and/or claims implies, unless otherwise stated, that the limits of the ranges are included.

The invention relates to a composition in the form of a concentrate that can be diluted in water, comprising:
a) at least one polyoxyethylene sorbitan fatty acid ester;
b) a lipophilic and water-dispersible semiochemical composition, in particular a semiochemical composition comprising bis(2-ethylhexyl)adipate and 1-isopropyl-2,2-dimethyltrimethylene diisobutyrate in a content of at least 40% by weight of the composition in the form of a concentrate,
the total sum of the compounds present in the composition in the form of a concentrate being equal to 100%.

In a particular embodiment of the invention, said semiochemical composition comprises from 40% to 60% by weight of bis(2-ethylhexyl)adipate and from 40% to 60% by weight of 1-isopropyl-2,2-dimethyltrimethylene diisobutyrate, the total sum of the compounds present in the semiochemical composition being equal to 100%.

In a preferred embodiment of the invention, said semiochemical composition comprises 50% by weight of bis(2-ethylhexyl)adipate and 50% by weight of 1-isopropyl-2,2-dimethyltrimethylene diisobutyrate.

The composition according to the invention is in the form of a concentrate that can be diluted in water, in particular water supplied by the end user. In particular, the composition is in the form of a concentrate, in particular in the form of a homogeneous and liquid mixture, preferably in the form of a yellow or brown concentrate of medium viscosity.

In the context of the invention, the term “concentrate” or the expression “in the form of a concentrate” refers to a composition in the form of a concentrate which can be the subject of further processing, in particular dilution, in particular in water, in order to obtain a so-called “ready-to-use” composition. The composition is said to be “in the form of a concentrate” to mean that its content of semiochemical composition is greater than the content of said semiochemical composition normally used in animals or humans in a non-therapeutic application.

In a particular embodiment of the invention, said at least one polyoxyethylene sorbitan fatty acid ester (also called ester of fatty acid and polyoxyethylene sorbitan; or ethoxylated sorbitan, sorbitan ester) includes in particular esters of C₈-C_{24,} preferably C₁₆-C_{22,} fatty acids and of polyoxyethylene sorbitan which may comprise from 1 to 150 oxyethylenated groups, such as, for example, polysorbate 20 in particular marketed under the name Tween 20® by CRODA, polysorbate 21 in particular marketed under the name Tween 21® by CRODA, polysorbate 40 in particular marketed under the name Tween 40® by CRODA, polysorbate 60 in particular marketed under the name Tween 60® by CRODA, polysorbate 61 in particular marketed under the name Tween 61® by CRODA, polysorbate 65 in particular marketed under the name Tween 65® by CRODA, polysorbate 80 in particular marketed under the name Tween 80® by CRODA, polysorbate 81 in particular marketed under the name Tween 81® by CRODA and polysorbate 85 in particular marketed under the name Tween 85® by CRODA.

In a particular embodiment of the invention, the composition in the form of a concentrate comprises a single polyoxyethylene sorbitan fatty acid ester, or a mixture of polyoxyethylene sorbitan fatty acid esters as described above.

In a preferred embodiment of the invention, the composition in the form of a concentrate comprises a single polyoxyethylene sorbitan fatty acid ester, in particular polysorbate 85 (also known as polyoxyethylene sorbitan trioleate) in particular marketed under the name Tween 85® by CRODA.

In a particular embodiment of the invention, said at least one polyoxyethylene sorbitan fatty acid ester is present in an amount ranging from 25% to 60% by weight, preferably from 30% to 40% by weight, even more preferably from 34.90% or 35% by weight, relative to the total weight of the composition in the form of a concentrate.

The composition according to the invention also comprises a semiochemical composition as active agent, as defined above. A particular semiochemical composition is described above and in the preferred embodiments and experimental examples that follow. The semiochemical compositions contained in the compositions according to the invention are in particular intended for non-therapeutic use, for example use for their behavioural or protective effect against pests (parasites, insects, etc.) on the animals or humans to which they are administered or on animals interacting with the latter, in the form of a composition diluted from the composition in the form of a concentrate according to the invention.

According to a particular embodiment, the semiochemical composition may be present in the composition in the form of a concentrate in an amount ranging from 60% to 70% by weight, preferably 65% by weight of semiochemical composition, relative to the total weight of the composition in the form of a concentrate.

The composition according to the invention may also comprise at least one additive provided that it does not interfere with the desired properties of the composition in the form of a concentrate.

In a particular embodiment of the invention, said at least one additive is selected from the group consisting of a preservative, in particular an antioxidant agent, a perfume, a masking agent, and mixtures thereof.

In the context of the invention, the term “preservative” refers to an agent that protects the composition according to the invention from changes in its physical or chemical state, its colour or its odour, or protects it from microbial contamination or degradation, for example by inhibiting the development of micro-organisms during the time the composition according to the invention is stored or used. Such an agent must be compatible with use of the composition according to the invention in contact with an animal or a human, and is advantageously non-irritating to the skin and eyes.

Examples of preservatives are known to the skilled in the art and include benzyl alcohol, didecyldimonium chloride, caprylyl glycol, phenylpropanol, sodium benzoate, phenyl acetate, sorbic acid, dehydroacetic acid, potassium sorbate, phenoxyethanol, cetrimonium bromide, triclosan, parabens and thiazolinones.

In the context of the invention, the term “antioxidant agent” refers to a preservative capable of inhibiting reactions promoted by oxygen, thereby preventing oxidation.

Examples of antioxidant agents are known to those skilled in the art and include in particular cinnamic acids and derivatives their such as pentaerythrityl tetra-di-t-butyl hydroxyhydrocinnamate in particular marketed under the name Tinogard TT® by BASF, vitamin A (retinol) and its derivatives (retinal, retinoic acid), vitamin C (ascorbic acid) and its derivatives (ascorbate), vitamin E (tocopherol) and its derivatives (tocopheryl), preferably Tinogard TT®. The composition according to the invention may comprise from 0% to 2% by weight, preferably from 0.02% to 0.3% by weight, even more preferably 0.1% by weight of antioxidant agent, relative to the total weight of the composition in the form of a concentrate.

In the context of the invention, the term “perfume” or perfuming agent refers to any hydrophobic substance designating a pure body or a mixture that is insoluble or sparingly soluble in water and capable of giving off a pleasant odour.

The composition according to the invention may comprise from 0% to 3% by weight, preferably from 0.2% to 2.5% by weight, even more preferably 1% or 2% by weight of perfume, relative to the total weight of the composition.

In the context of the invention, the term “masking agent” refers to an agent that reduces or inhibits the basic odour or taste of the product.

Examples of masking agents are known to the skilled in the art and include in particular beta sitosterol, citric acid, bisabolol and calendula officienalis flower extract.

The composition according to the invention can comprise from 0% to 3% by weight, preferably from 0.2% to 2.5% by weight, even more preferably 1% or 2% by weight of a masking agent, relative to the total weight of the composition.

In a preferred embodiment of the invention, the composition in the form of a concentrate comprises:
a) sorbitan polyoxyethylene trioleate;
b) a semiochemical composition comprising bis(2-ethylhexyl) adipate and 1-isopropyl-2,2-dimethyltrimethylene diisobutyrate, in particular in an amount at least equal to 40% by weight of the composition in the form of a concentrate; and
c) pentaerythrityl tetra-di-t-butyl hydroxyhydrocinnamate, the total sum of the compounds present in the composition in the form of a concentrate being equal to 100%.

In an even more preferred embodiment of the invention, the composition in the form of a concentrate comprises:
a) from 25% to 60% by weight, preferably from 30% to 40% by weight, even more preferably from 34.9% or 35% by weight, expressed by weight relative to the total weight of the composition in the form of a concentrate, of sorbitan polyoxyethylene trioleate;
b) from 60% to 70% by weight, preferably 65% by weight, expressed by weight relative to the total weight of the composition in the form of a concentrate, of a semiochemical composition comprising bis(2-ethylhexyl)adipate and 1-isopropyl-2,2-dimethyltrimethylene diisobutyrate, in particular from 40% to 60% by weight of bis(2-ethylhexyl)adipate and from 40% to 60% by weight of 1-isopropyl-2,2-dimethyltrimethylene diisobutyrate, preferably 50% by weight of bis(2-ethylhexyl)adipate and 50% by weight of 1-isopropyl-2,2-dimethyltrimethylene diisobutyrate; and
c) from 0.02% to 0.3% by weight, expressed by weight relative to the total weight of the composition in the form of a concentrate, of pentaerythrityl tetra-di-t-butyl hydroxyhydrocinnamate.

In a particular embodiment of the invention, the composition in the form of a concentrate has a Brookfield viscosity of between 5 mPa·s and 200 mPa·s, in particular of between 10 mPa·s and 100 mPa·s, as measured at 25° C. The viscosity within the meaning of the present invention is a Brookfield viscosity measured at room temperature (25° C.), preferably after 24 hours, by means of a viscometer, for example a Brookfield digital viscometer (model LDVI Prime), device S 61 and rotation speed 100 RPM.

In a particular embodiment of the invention, the composition in the form of a concentrate has a pH of between 7.0 and 11.0, preferably a pH of between 7.5 and 10.0.

In a particular embodiment of the invention, the composition diluted in water according to the invention has a pH of between 5.0 and 8.0, preferably a pH of between 7.0 and 8.0. Preferably, the diluted composition has a pH value compatible with topical, in particular cutaneous, application of the composition to animal or human skin.

The invention also relates to a composition diluted in water, comprising:
a) a composition in the form of a concentrate according to the invention; and
b) water.

This dilution can be carried out by an end user (in particular extemporaneously) in any location, in any quantities, for example in small-scale or industrial-scale quantities (such as livestock buildings).

In a particular embodiment of the invention, the composition diluted in water is in the form of an aqueous suspension, in particular an oil-in-water (O/W) emulsion, preferably in the form of a homogeneous mixture.

In the context of the invention, the term “emulsion” refers to a mixture of two immiscible liquid substances, such as oil and water. In an oil-in-water (O/W) emulsion, the oil is dispersed in the water in the form of droplets, particularly small droplets.

In the context of the invention, the term “homogeneous mixture” refers to a mixture in which the individual constituents cannot be distinguished by the naked eye.

In a preferred embodiment of the invention, the composition diluted in water comprises:
a) 5% (v%/v%) of said composition in the form of a concentrate having a content ranging from 60% to 70% by weight, preferably 65% by weight, of the semiochemical composition; and
b) 95% (v%/v%) of water.

In another particular embodiment of the invention, the composition in the form of a concentrate according to the invention is diluted in water with a dilution factor equal to 1/20.

Preferably, said composition in the form of a concentrate has a content ranging from 60% to 70% by weight, preferably 65% by weight of the semiochemical composition, and/or the composition diluted in water has a content ranging from 2% to 5% by weight, preferably from 3 to 3.5% by weight, preferably 3.25% by weight of the semiochemical composition.

The invention also relates to a process for preparing a composition in the form of a concentrate according to the invention, comprising the following steps:
a) the semiochemical composition, said at least one polyoxyethylene sorbitan fatty acid ester and optionally said at least one additive are mixed, with stirring and at a temperature of between 65° C. and 75° C., preferably at a temperature of 70° C., so as to obtain a homogeneous mixture;
b) optionally, a perfume is added;
c) the composition obtained in step a) or b) is recovered in the form of a concentrate.

In a particular embodiment of the process according to the invention, at the end of step a), the composition in the form of a concentrate is cooled to room temperature.

The invention also relates to a process for preparing a composition diluted in water, comprising a step of diluting in water the composition in the form of a concentrate according to the invention, in particular with a dilution factor equal to 1/20.

According to another aspect, the invention relates to a non-therapeutic use of a composition diluted in water according to the invention. According to this use, the composition diluted in water according to the invention is used to obtain the semiochemical effect(s) expected from the semiochemical composition it contains. Thus, a composition diluted in water according to the invention is used to improve the management of the behaviour or interactions of target animals or even their well-being, by transmitting information or signals vectored by semiochemical agents.

One use of the composition diluted in water according to the invention is, for example, as a tool or means of improving animal husbandry. One use of the composition diluted in water according to the invention is, for example, as an adaptive tool or means for the animal in relation to its environment.

The use of the composition diluted in water according to the invention comprises topical, in particular cutaneous, administration of said composition to an animal, in particular a mammal, or to a human, or to animals treated as a group.

In a particular embodiment of the invention, the composition diluted in water is administered topically, in particular cutaneously, to the skin of an animal or to human skin.

In a particular embodiment according to the invention, the animal is a domestic animal or a farm animal, in particular a mammal, the animal being preferably selected from the group consisting of pets such as rabbits, cats, dogs, birds or from the group of farm animals such as horses or production animals such as pigs, cattle, poultry, for example hens.

In a particular embodiment, the invention relates to a non-therapeutic use of a composition diluted in water according to the invention, for topical, in particular cutaneous, administration to poultry such as hens, in particular on the skin of a poultry such as a hen, in particular to prevent infestation by parasites such as the poultry red mite (Dermanyssus sp. or Ornithonyssus sylviarum).

In another particular embodiment, the invention relates to a non-therapeutic use of a composition diluted in water according to the invention, for topical, in particular cutaneous, administration to an animal, for example birds or mammals, in particular on the skin of birds or mammals, or to a human, in particular on human skin, in particular to prevent infestation or bites by haematophagous invertebrates, in particular haematophagous insects, such as mosquitoes, mites or ticks.

In a particular embodiment according to the invention, the composition diluted in water comprises a composition in the form of a concentrate according to the invention comprising a semiochemical composition and water, said semiochemical composition comprising bis(2-ethylhexyl) adipate and 1-isopropyl-2,2-dimethyltrimethylene diisobutyrate, in particular from 40% to 60% by weight of bis(2-ethylhexyl) adipate and from 40% to 60% by weight of 1isopropyl-2,2-dimethyltrimethylene diisobutyrate, preferably 50% by weight of bis(2-ethylhexyl) adipate and 50% by weight of 1-isopropyl-2,2-dimethyltrimethylene diisobutyrate; and relates to the non-therapeutic use of said composition diluted in water for topical, in particular cutaneous, administration to poultry such as hens, in particular on the skin of poultry such as hens, in particular to prevent infestation by parasites such as the poultry red mite (Dermanyssus sp. or Ornithonyssus sylviarum).

In another particular embodiment according to the invention, the composition diluted in water comprises a composition in the form of a concentrate according to the invention comprising a semiochemical composition and water, said semiochemical composition comprising bis(2-ethylhexyl) adipate and 1-isopropyl-2,2-dimethyltrimethylene diisobutyrate, in particular from 40% to 60% by weight of bis(2-ethylhexyl) adipate and from 40% to 60% by weight of 1-isopropyl-2,2-dimethyltrimethylene diisobutyrate, preferably 50% by weight of bis(2-ethylhexyl) adipate and 50% by weight of 1-isopropyl-2,2-dimethyltrimethylene diisobutyrate; and relates to the non-therapeutic use of said composition diluted in water for topical, in particular cutaneous, administration to an animal, for example birds or mammals, in particular on the skin of birds or mammals, or to a human, in particular on human skin, in particular to prevent infestation or bites by haematophagous invertebrates, in particular haematophagous insects, such as mosquitoes, mites or ticks.

Preferably, in the context of the uses mentioned above, the composition diluted in water according to the invention comprises: a) 5% (v%/v%) of the composition in the form of a concentrate according to the invention having a content ranging from 60% to 70% by weight, preferably 65% by weight of the semiochemical composition; and b) 95% (v%/v%) of water; and/or the composition in the form of a concentrate is diluted in water with a dilution factor equal to 1/20.

The above-mentioned uses of the composition diluted in water according to the invention are non-therapeutic uses, and exclude any therapeutic use practised on the animal or human body.

### EXAMPLES

The following examples are given by the way of illustration and therefore should not be construed to limit the scope of the invention.

### Example 1. Concentrate That Can be Diluted in Water

Compositions in the form of concentrate that can be diluted with water according to the invention having the composition shown in the following Table 1 have been prepared.

TABLE 1

| | | | Percentage |
|---|---|---|---|
| Compound name | INCI | CAS No. | by weight |
| Semiochemical | / | / | 65% |
| composition CS1 | | | |
| Polysorbate 85 | POLYSORBATE 85 | 9005-70-3 | 34.9% |
| Tinogard TT | PENTAERYTHRITYL | 6683-19-8 | 0.1% |
| | TETRA-DI-T-BUTYL | | |
| | HYDROXYHYDRO- | | |
| | CINNAMATE | | |

### Semiochemical Composition CS1 Used as an Example in the Formulation of Table 1

CS1: 50% by weight of bis(2-ethylhexyl) adipate and 50% by weight of 1-isopropyl-2,2-dimethyltrimethylene diisobutyrate.

### Example 2. Process for the Preparation of the Concentrate That can be Diluted in Water According to Example 1

The concentrates that can be diluted in water according to Example 1 were prepared according to the following protocol:
1. The semiochemical composition CS1, Tinogard TT and Polysorbate 85 were weighed and added into a container adapted to contain the final volume of the mixture.
2. The mixture was then stirred and heated to 70° C. until a homogeneous mixture was obtained.
3. The composition was recovered in the form of a concentrate.

The composition, in the form of a concentrate that can be diluted in water, was orange or brown in colour and of medium viscosity.

### Example 3. Dilution of the Composition in the Form of a Concentrate

For the use of the composition prepared in the form of a concentrate according to Example 1, a dilution is carried out, for example by the end user. If the final diluted composition is composed of 3.25% of semiochemical composition, for example CS1, the dilution to be made is a dilution by 20 since the concentrate is at 65% of semiochemical composition such as CS1. Table 2 below shows examples of dilution for different volumes.

TABLE 2

| | |
|---|---|
| Components | Dilution by 20 |

| | | | | |
|---|---|---|---|---|
| Concentrate according | 2.5 | 5 | 10 | 25 |
| to Example 1 (65%) (L) | | | | |
| Water (I) | 47.5 | 95 | 190 | 475 |
| Final mixture (3.25%) | 50 L | 100 L | 200 L | 500 L |

For example, a 5 L can of composition in the form of a concentrate is used for a final volume of 100 L.

### Example 4. Organoleptic Measurements of the Composition in the Form of a Concentrate According to Example 1

3 criteria (appearance, colour, odour) were selected for organoleptic measurements.
Appearance: Liquid
Colour: Yellow/brown
Odour: Characteristic odour.

### Example 5. pH Measurements of the Composition in the Form of a Concentrate According to Example 1

The pH measurements were carried out using a LE 438 IP 67 electrode marketed by Mettler Toledo®. This is a 2-in-1 glass pH electrode with integrated temperature sensor and liquid electrolyte for rapid measurements in the laboratory; for aqueous samples such as acids, bases, etc.

The specifications for this electrode are given in Table 3 below.

TABLE 3

| | |
|---|---|
| Measuring range | pH 0-14 |
| Zero point | 7.00 ± 0.25 |
| Temperature range | 0-80° C. |
| Temperature sensor | NTC 30 K Ω |
| Membrane resistance | <250 MΩ |
| (25° C.) | |
| Reference electrolyte | 3 mol/L KCl solution |
| | saturated with AgCl |
| Reference system | Ag/AgCl |
| Reference diaphragm | Gel |

Before any measurements are taken, the pH meter used is calibrated. The nature of the semiochemical composition can affect the pH of the emulsion, as some semiochemicals have an acid pH. In the context of a topical application, particularly for the skin, it is therefore necessary to measure the pH of each composition obtained and, if necessary, to adjust the pH.

pH measurements were carried out after obtaining the composition in the form of a concentrate, or the composition diluted in water according to the invention, and are reported below:
Concentrate at 65%: 7.5 <pH<10.0
Diluted composition (dilution factor 1/20): 5.5<pH<7.5.

### Example 6. Measurement of the Viscosity of the Composition in the Form of a Concentrate According to Example 1

Viscosity is the resistance of a fluid to sliding from one layer to another. The viscosity of each composition must be checked after each production run. Viscosity is influenced by a number of factors, including temperature, the measurement device used, the measurement speed used and the time of measurement during production (measurements at T0 and T+24 h, T+48 h). The viscosity should be measured when the solution is at room temperature, preferably at T+24 h.

The viscosity of the composition in the form of a concentrate according to Example 1 was measured using a Brookfield digital viscometer (model DV-I Prime), device S 61 and at a rotation speed of 100 RPM.

The Brookfield DV-I Prime viscometer measures the viscosity of fluids at a given velocity gradient. The principle of operation involves rotating a moving part (which is immersed in the liquid to be tested) by means of a calibrated spring. The measurement range (in centipoises or milliPascal-second) is determined by the speed of rotation of the device, the size and shape of the device, the cross-section where the device is rotating and the full scale of the torque of the calibrated spring.

The specifications of the Brookfield DV-I Prime viscometer are given in Table 4 below.

TABLE 4

| | |
|---|---|
| Speeds (rpm) | 0.0; 0.3; 0.6; 1.5; 3; 6; 12; 30; 60; |
| | 0.5; 1; 2; 2.5; 4; 5; 10; 20; 50; 100 |
| Viscosity range (cP) | 15-2,000,000 |
| Operating medium | Temperature range from 0° C. to |
| | 40° C. (32° F. to 104° F.), 20% to |
| | 80% R.H.: non-condensing |
| Analogue torque output | 0-1 Vdc (torque from 0 to 100%) |
| Accuracy of viscosity | ±1° C.: -of full scale |
| measurement | |
| Reproducibility of | 0.2% of full scale used |
| viscosity measurement | |
| Devices used | S61; S62; S63; S64 |

As a result of these measurements, the composition in the form of a concentrate according to Example 1 had a viscosity of between 10 and 100 cP or mPa·s.

### References

Silverstein, R.M., 1977. “Complexity, diversity and specificity of behavior-modifying chemicals; examples from Coleoptera and Hymenoptera,” Chemical Control of Insect Behavior: Theory and Application. Publishers H.H. Shorey & J.J.Mckelvey pp. 231-251 New York: Plenum Press);
Wyatt T.D., 2003 “Pheromones and Animal Behavior. Communication by smell and taste, pp. 1-4 New York: Cambridge University Press.

1. A composition, which is in the form of a concentrate that can be diluted in water, and comprises:
a) at least one polyoxyethylene sorbitan fatty acid ester; and
b) a lipophilic and water-dispersible semiochemical composition comprising bis(2-ethylhexyl)adipate and 1-isopropyl-2,2-dimethyltrimethylene diisobutyrate in a content of at least 40% by weight of the composition in the form of a concentrate, the total sum of the compounds present in the composition in the form of a concentrate being equal to 100%.

2. The composition according to claim 1, wherein the semiochemical composition comprises from 40% to 60% by weight of bis(2-ethylhexyl) adipate and from 40% to 60% by weight of 1-isopropyl-2,2-dimethyltrimethylene diisobutyrate, the total sum of the compounds present in the semiochemical composition being equal to 100%.

3. The composition according to claim 1, wherein the semiochemical composition comprises 50% by weight of 2-bis(hexylethyl)adipate and 50% by weight of 1-isopropyl-2,2-dimethyltrimethylene diisobutyrate.

4. The composition according to claim 1, wherein the semiochemical composition is present in an amount ranging from 60% to 70% by weight, relative to the total weight of the composition in the form of a concentrate.

5. The composition according to claim 4, wherein the semiochemical composition is present in an amount of 65% by weight, relative to the total weight of the composition in the form of a concentrate.

6. The composition according to claim 1, wherein the at least one polyoxyethylene sorbitan fatty acid ester is present in an amount ranging from 25% to 60% by weight, relative to the total weight of the composition in the form of a concentrate.

7. The composition according to claim 1, wherein it additionally comprises at least one additive selected from the group consisting of a preservative, a perfume, a masking agent, and mixtures thereof.

8. The composition according to claim 7, wherein the preservative is an antioxidant agent selected from the group consisting of cinnamic acids, pentaerythrityl tetra-di-t-butyl hydroxyhydrocinnamate, vitamin A, retinal, retinoic acid, vitamin C, ascorbate, vitamin E and tocopheryl.

9. The composition according to claim 8, wherein the antioxidant agent is pentaerythrityl tetra-di-t-butyl hydroxyhydrocinnamate.

10. The composition according to claim 1, wherein the antioxidant agent is present in an amount ranging from 0.02% to 0.3% by weight, relative to the total weight of the composition in the form of a concentrate.

11. The composition according to claim 1, wherein it comprises:
a) sorbitan polyoxyethylene trioleate;
b) a semiochemical composition comprising bis(2-ethylhexyl) adipate and 1-isopropyl-2,2-dimethyltrimethylene diisobutyrate; and
c) pentaerythrityl tetra-di-t-butyl hydroxyhydrocinnamate.

12. The composition according to claim 11, wherein it comprises:
a) from 25% to 60% by weight of sorbitan polyoxyethylene trioleate;
b) from 60% to 70% by weight of the semiochemical composition comprising bis(2-ethylhexyl) adipate and 1-isopropyl-2,2-dimethyltrimethylene diisobutyrate; and
c) from 0.02% to 0.3% by weight of pentaerythrityl tetra-di-t-butyl hydroxyhydrocinnamate, the percentages by weight being relative to the total weight of the composition in the form of a concentrate.

13. The composition diluted in water, comprising:
a) the composition in the form of a concentrate according to claim 1; and
b) water.

14. The composition diluted in water according to claim 13, comprising:
a) 5% (v%/v%) of the composition in the form of a concentrate having a content ranging from 60% to 70% by weight of the semiochemical composition; and
b) 95% (v%/v%) of water.

15. The composition diluted in water according to claim 13, wherein the composition in the form of a concentrate is diluted in water with a dilution factor equal to 1/20.

16. A process for preparing the composition in the form of a concentrate according to claim 1, comprising the following steps:
a) the semiochemical composition, the at least one polyoxyethylene sorbitan fatty acid ester and optionally the at least one additive are mixed, with stirring and at a temperature of between 65° C. and 75° C., so as to obtain a homogeneous mixture;
b) optionally, a perfume is added;
c) the composition obtained in step a) or b) is recovered in the form of a concentrate.

17. The process according to claim 16, wherein, at the end of step a), the composition in the form of a concentrate is cooled to room temperature.

18. Non-therapeutic use of the composition diluted in water according to claim 13, wherein the use comprises topical administration to poultry to prevent infestation by parasites.

19. The non-therapeutic use according to claim 18, wherein the parasites are the poultry red mite (Dermanyssus sp. or Ornithonyssus sylviarum).

20. Non-therapeutic use of the composition diluted in water according to claim 13, wherein the use comprises topical administration to an animal or to a human to prevent infestation or bites by haematophagous invertebrates.

21. The non-therapeutic use according to claim 20, wherein the haematophagous invertebrates are selected from the group consisting of mosquitoes, mites and ticks.
